(19) United States
(12) Patent Application Publication
(10) Pub. No.: US 2024/0156709 A1
(43) Pub. Date: May 16, 2024

(54) SYSTEMS AND METHODS FOR STABILIZING COMPOSITIONS AND COMPOSITIONS COMPRISING THE SYSTEMS

(71) Applicant: L 'Oreal, Paris (FR)

(72) Inventors: Kazumitsu KAWAKAMI, Westfield, NJ (US); Liliana XAVIER, Elizabeth, NJ (US)

(21) Appl. No.: 17/977,135

(22) Filed: Oct. 31, 2022

(51) Int. Cl. A61K 8/73; A61K 8/34; A61K 8/81; A61Q 5/00; A61Q 5/12

(52) U.S. Cl. CPC A61K 8/732; A61K 8/342; A61K 8/817; A61Q 5/002; A61Q 5/12

(57) ABSTRACT

The disclosure relates to systems for stabilizing compositions, methods for stabilizing compositions using the systems, and compositions comprising the systems. The systems comprise at least one starch polymer, at least one cationic polymer, and at least one fatty alcohol. The compositions may include at least one electrolyte, and may be compositions for treating keratinous substrates.

### TECHNICAL FIELD

The present disclosure relates to systems for stabilizing compositions, methods of stabilizing compositions with the systems, and compositions comprising the systems.

### BACKGROUND

Consumers desire compositions for treating, caring for, conditioning, making up, etc., keratinous substrates such as skin, hair, and nails. However, formulating compositions that can satisfactorily address these needs is challenging. For example, formulating compositions that include the active ingredient(s) necessary to address the particular concern (e.g. reduce wrinkles, repair damaged hair, etc.) while at the same time providing compositions that are considered to have the qualities the consumer desires (e.g. not too runny, not too thick, etc.) as well as the quality needed to ensure consumer confidence in the product (e.g. the composition does not separate over time) is difficult.

For example, consumers desire compositions to condition or repair damaged hair, which can also provide cosmetic benefits such as volume and shine. Conditioning the hair can help manage hair that is dry, frizzy, and/or prone to tangles which can result from various factors such as environmental conditions and/or repeated styling, e.g. with heat and/or styling tools.

Further, processes to alter the color or shape of the hair, including those using harsh chemicals such as bleaching, dyeing, straightening, and permanent waving, can damage the hair fiber. Such damage may appear as split ends, broken hair, flyaway hair, etc., and may cause the hair to feel rough and look dull and unhealthy.

Hair care products are used to address these concerns. For example, a hair treatment or conditioner composition can be used on hair in order to confer conditioning, smoothing, and/or softening properties to the hair. However, certain components that may be advantageous to include in such hair treatment and/or conditioner compositions due to the beneficial properties they can impart, most notably electrolytes, are known to cause the composition to be unstable.

Thus, the present disclosure relates to systems for stabilizing compositions for treatment of keratinous substrates, methods for stabilizing compositions using the systems, and compositions comprising the systems.

### SUMMARY

It has been surprisingly discovered that systems described herein can be used to stabilize compositions, for example compositions for treating keratinous substrates, caring for keratinous substrates, conditioning keratinous substrates, etc., such as keratin fibers including hair. The stability provided may, in various embodiments, be long-lasting.

In some embodiments, the disclosure relates to systems for stabilizing compositions, which compositions may optionally include at least one electrolyte in an amount of at least about 0.25%, such as at least about 0.5%, at least about 0.75%, or at least about 1%. The compositions may, in various embodiments, be compositions for treating keratin substrates, e.g. keratin fibers such as hair. The systems comprise a synergistic combination of at least one starch, at least one cationic polymer, and at least one fatty alcohol, which are present in amounts sufficient to stabilize the composition.

In various embodiments, the starch polymers, which may be modified or unmodified, can be chosen from corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures of two or more thereof, and in some embodiments comprises, consists essentially of, or consists of modified or unmodified potato starch. The total amount of starch polymers, relative to the weight of the composition comprising the system, may, in various embodiments, be at least about 0.75%, such as at least about 0.8%, at least about 0.85%, at least about 0.9%, at least about 0.95%, or at least about 1%. In some embodiments, the total amount of starch polymers is not less than 1% by weight, based on the weight of the composition comprising the system.

In some embodiments, the cationic polymers may be chosen from cationic quaternary ammonium polymers. As non-limiting examples, Polyquaternium-6, Polyquaternium-15, Polyquaternium-22, Polyquaternium-32, Polyquaternium-37, Polyquaternium-39, Polyquaternium-47, Quaternium-14, Quaternium-22, Quaternium-24, or mixtures of two or more thereof may be chosen. In some embodiments, the cationic polymer comprises, consists essentially of, or consists of Polyquaternium-37. The total amount of cationic polymers, relative to the weight of the composition comprising the system, may, in various embodiments, be at least about 0.35%, such as at least about 0.4%, at least about 0.45%, or at least about 0.5%. In some embodiments, the total amount of cationic polymers is not less than 0.5% by weight, based on the weight of the composition comprising the system.

It may, in some embodiments, be advantageous to choose amounts of starch polymers and cationic polymers so that a weight ratio of the total amount of starch polymers to the total amount of cationic polymers is greater than 1, such as greater than about 1.25, greater than about 1.5, greater than about 1.75, greater than about 2, greater than about 2.25, greater than about 2.5, greater than about 2.75, or greater than about 3, such as ranging from greater than 1 up to about 3, up to about 4, or up to about 5.

In various embodiments, the fatty alcohols, which may be liquid or solid, are not alkoxylated or glycerolated and comprise at least 8 carbon atoms. By way of example, octyldodecanol, cetyl alcohol, stearyl alcohol, cetearyl alcohol, behenyl alcohol, lauryl alcohol, myristic or myristyl alcohol, arachidyl alcohol, lignoceryl alcohol, or mixtures of two or more thereof may be chosen. In some embodiments, the fatty alcohol comprises, consists essentially of, or consists of octyldodecanol, cetearyl alcohol, cetyl alcohol, stearyl alcohol, or mixtures of two or more thereof.

In some embodiments, the disclosure relates to stable compositions comprising the systems described herein. The compositions may optionally include at least one electrolyte, for example in an amount of at least about 0.25%, such as at least about 0.5%, at least about 0.75%, or at least about 1%. The electrolytes may include an anion such as, for example, phosphate, chloride, sulfate, or citrate, and a cation such as, for example, sodium, ammonium, potassium, or magnesium. The compositions comprise total amounts of starch polymers, cationic polymers, and fatty alcohols sufficient to stabilize the compositions.

The compositions may optionally be compositions for treating keratinous substrates, for example treating, caring for, and/or conditioning keratin fibers such as hair. As such, the stable compositions may optionally comprise one or more additional components typically found in such compositions, for example surfactants such as anionic, cationic, nonionic, and/or amphoteric surfactants; solvents, such as water and/or non-aqueous solvents; fatty compounds such as oils; thickening agents; and/or cosmetically-acceptable adjuvants.

In some embodiments, the stable compositions comprise: at least one starch polymer, which optionally comprises modified or unmodified potato starch, and the total amount of starch polymer(s) is at least about 0.75%; at least one cationic polymer, which optionally comprises Polyquaternium-37, and the total amount of cationic polymers is at least about 0.35%; at least one fatty alcohol, which optionally comprises octyldodecanol, cetearyl alcohol, cetyl alcohol, stearyl alcohol, or mixtures of two or more thereof, and the total amount of fatty alcohols ranges from about 0.5% to about 10%; at least one electrolyte, and the total amount of electrolytes is at least about 0.25%, such as at least about 0.5%, at least about 0.75%, or at least about 1%; at least one surfactant, which may optionally be chosen from cationic and/or nonionic surfactants; and water, where all amounts are by weight, based on the weight of the stable composition, and where the weight ratio of the total amount of starch polymers to the total amount of cationic polymers is greater than 1, for example up to about 5, up to about 4, or up to about 3.

In some embodiments, the stable compositions comprise: at least one starch polymer, which optionally comprises modified or unmodified potato starch, and the total amount of starch polymer(s) is at least about 1%, e.g. not less than 1%; at least one cationic polymer, which optionally comprises Polyquaternium-37, and the total amount of cationic polymers is at least about 0.5%, e.g. not less than 0.5%; at least one fatty alcohol, which optionally comprises octyldodecanol, cetearyl alcohol, cetyl alcohol, stearyl alcohol, or mixtures of two or more thereof, and the total amount of fatty alcohols ranges from about 0.5% to about 10%; at least one electrolyte, and the total amount of electrolytes is at least about 0.25%, such as at least about 0.5%, at least about 0.75%, or at least about 1%; at least one surfactant, which may optionally be chosen from cationic and/or nonionic surfactants; and water, where all amounts are by weight, based on the weight of the stable composition, and where the weight ratio of the total amount of starch polymers to the total amount of cationic polymers is greater than 1, for example up to about 5, up to about 4, or up to about 3.

The compositions may, in various embodiments, have a viscosity of less than about 30000 mPas, such as less than about 25000 mPas, less than about 20000 mPas, less than about 18000 mPas, less than about 15000 mPas, less than about 152000 mPas, less than about 10000 mPas, or less than about 8000 mPas.

In various embodiments, the compositions are stable for at least 4 weeks, and in some embodiments at least 8 weeks, at least 12 weeks, at least 24 weeks, at least 6 months, or at least 12 months or more, such as up to about 2 years.

In further embodiments, the disclosure relates to methods for stabilizing a composition by including a system according to the disclosure therein. The compositions which can be stabilized can optionally include at least one electrolyte, for example in an amount of at least about 0.25%, such as at least about 0.5%, at least about 0.75%, or at least about 1%. The compositions, in at least some embodiments, are compositions for treating keratinous substrates, for example for treating, caring for, and/or conditioning keratin fibers, such as hair.

### DETAILED DESCRIPTION

The disclosure relates to systems for stabilizing compositions that can be used for treating keratinous substrates, such as keratin fibers, in particular compositions comprising at least one electrolyte. Compositions which comprise at least one electrolyte can provide strength and/or repair to keratinous substrates such as skin, hair, and nails, that have been damaged. However, such compositions are generally unstable and efforts to stabilize such compositions have had limited success. By including the systems described herein, however, the compositions are surprisingly stable. Therefore, the combination of components disclosed for the systems herein provides surprising and unexpected stability to compositions that can be used for treating keratinous substrates, such as keratin fibers, particularly when the compositions comprise one or more electrolytes.

The disclosure also relates to methods for stabilizing compositions using the systems and stable compositions comprising the systems.

### I. Systems

In various embodiments, the systems comprise a synergistic combination of (a) at least one starch polymer, (b) at least one cationic polymer, and (c) at least one fatty alcohol.

### Starch Polymers

Systems according to the disclosure comprise at least one polymer derived from starch. Optionally, the systems may, in some embodiments, comprise at least two, at least three, at least four, etc., starch polymers.

Starches are nonionic polymers that can be modified to be cationic or anionic. Nonionic, cationic, or anionic starch polymers can be used in systems according to the disclosure, as well as mixtures thereof.

Non-limiting examples of starches that can be used include starches of cereals, starches of tubers, starches of beans, starches of wild grasses, stem or trunk starches, and modified starches thereof. For example, in various embodiments these starches may by modified or unmodified and may be chosen from corn starch, wheat starch, rice starch, waxy corn starch, oat starch, cassava starch, waxy barley, waxy rice starch, glutenous rice starch, sweet rice starch, amioca, potato starch, tapioca starch, oat starch, sago starch, sweet rice, or mixtures of two or more thereof. In at least some embodiments, the starch polymer comprises, consists essentially of, or consists of one or more of corn starch, tapioca starch, potato starch, sweet potato starch, sago starch, modified starches thereof, or mixtures thereof. In further embodiments, the starch polymer comprises, consists essentially of, or consists of one or more of potato starch, sweet potato starch, modified starches thereof, or mixtures thereof. In at least some embodiments, the starch polymer comprises, consists essentially of, or consists of modified potato starch.

Modified starches are typically obtained by subjecting the starch to a derivatization treatment (e.g., etherification, esterification, alkylation, or grafting), a decomposition treatment (e.g., roasting, enzymatic denaturation, oxidation, or an acid treatment), or a processing step (e.g., gelatinization, granulation, or pore introduction). In some embodiments, the modification includes cross-linking, stabilizing, phosphorylizing, or hydrolyzing the starch.

In some embodiments, the modified starch is included in the system in cationic form, such as in the form of hydrolyzed starch (e.g., acid, enzyme, or alkaline degradation), oxidized starch (e.g., peroxide, peracid, hypochlorite, alkaline, or any other oxidizing agent), physically/mechanically degraded starch (e.g., via thermo-mechanical energy input of processing equipment), or combinations thereof. Exemplary and non-limiting cationic starch polymers that can be used include degraded cationic maize starch, cationic tapioca, cationic potato starch, or mixtures thereof. In at least some embodiments, the starch polymer comprises, consists essentially of, or consists of cationic starch polymers. In further embodiments, the starch polymer comprises, consists essentially of, or consists of one or more of cationic maize starch, cationic potato starch, cationic sweet potato starch, or mixtures thereof.

The starch polymers are present in the systems in a total amount sufficient, in combination with the other components of the systems, to stabilize the composition in which the system is included. For example, the starch polymers can be present in a total amount of at least about 0.75%, such as at least about 0.8%, at least about 0.85%, at least about 0.9%, at least about 0.95%, or at least about 1% by weight, relative to the total weight of the composition in which the system is present. For example, the systems may comprise a total amount of starch polymers ranging from about 0.75% to about 10%, from about 0.8% to about 8%, from about 0.85% to about 6%, from about 0.9% to about 4%, from about 0.95% to about 3%, or from about 1% to about 2.5% by weight, relative to the total weight of the composition in which the system is present. In some embodiments, the total amount of starch polymers in the system is not less than 0.75%, not less than 0.8%, not less than 0.85%, not less than 0.9%, not less than 0.95%, or not less than 1% by weight, relative to the total weight of the composition in which the system is present.

In some embodiments, the system may comprise a total amount of starch polymers ranging from about 0.75% to about 10%, such as from about 0.75% to about 9%, from about 0.75% to about 8%, from about 0.75% to about 7%, from about 0.75% to about 6%, from about 0.75% to about 5%, from about 0.75% to about 4.5%, from about 0.75% to about 4%, from about 0.75% to about 3.5%, from about 0.75% to about 3%, from about 0.75% to about 2.75%, from about 0.75% to about 2.5%, from about 0.75% to about 2.25%, from about 0.75% to about 2%, from about 0.75% to about 1.75%, from about 0.75% to about 1.5%, from about 0.75% to about 1.25%, or from about 0.75% to about 1% by weight, relative to the total weight of the composition in which the system is present.

In some embodiments, the system comprises a total amount of starch polymers ranging from about 0.75% to about 2.5%, from about 0.75% to about 2.4%, from about 0.75% to about 2.3%, from about 0.75% to about 2.2%, from about 0.75% to about 2.1%, from about 0.75% to about 1.9%, from about 0.75% to about 1.8%, from about 0.75% to about 1.7%, from about 0.75% to about 1.6%, from about 0.75% to about 1.5%, from about 0.75% to about 1.4%, from about 0.75% to about 1.3%, from about 0.75% to about 1.2%, from about 0.75% to about 1.1%, from about 0.8% to about 5%, from about 0.8% to about 4%, from about 0.8% to about 3%, from about 0.8% to about 2.5%, from about 0.8% to about 2.4%, from about 0.8% to about 2.3%, from about 0.8% to about 2.2%, from about 0.8% to about 2.1%, from about 0.8% to about 1.9%, from about 0.8% to about 1.8%, from about 0.8% to about 1.7%, from about 0.8% to about 1.6%, from about 0.8% to about 1.5%, from about 0.8% to about 1.4%, from about 0.8% to about 1.3%, from about 0.8% to about 1.2%, from about 0.8% to about 1.1%, from about 0.8% to about 1%, from about 0.85% to about 5%, from about 0.85% to about 4%, from about 0.85% to about 3%, from about 0.85% to about 2.5%, from about 0.85% to about 2.4%, from about 0.85% to about 2.3%, from about 0.85% to about 2.2%, from about 0.85% to about 2.1%, from about 0.85% to about 1.9%, from about 0.85% to about 1.8%, from about 0.85% to about 1.7%, from about 0.85% to about 1.6%, from about 0.85% to about 1.5%, from about 0.85% to about 1.4%, from about 0.85% to about 1.3%, from about 0.85% to about 1.2%, from about 0.85% to about 1.1%, from about 0.85% to about 1%, from about 0.9% to about 5%, from about 0.9% to about 4%, from about 0.9% to about 3%, from about 0.9% to about 2.5%, from about 0.9% to about 2.4%, from about 0.9% to about 2.3%, from about 0.9% to about 2.2%, from about 0.9% to about 2.1%, from about 0.9% to about 1.9%, from about 0.9% to about 1.8%, from about 0.9% to about 1.7%, from about 0.9% to about 1.6%, from about 0.9% to about 1.5%, from about 0.9% to about 1.4%, from about 0.9% to about 1.3%, from about 0.9% to about 1.2%, from about 0.9% to about 1.1%, from about 0.9% to about 1%, from about 0.95% to about 5%, from about 0.95% to about 4%, from about 0.95% to about 3%, from about 0.95% to about 2.5%, from about 0.95% to about 2.4%, from about 0.95% to about 2.3%, from about 0.95% to about 2.2%, from about 0.95% to about 2.1%, from about 0.95% to about 1.9%, from about 0.95% to about 1.8%, from about 0.95% to about 1.7%, from about 0.95% to about 1.6%, from about 0.95% to about 1.5%, from about 0.95% to about 1.4%, from about 0.95% to about 1.3%, from about 0.95% to about 1.2%, from about 0.95% to about 1.1%, or from about 0.95% to about 1% by weight, relative to the total weight of the composition in which the system is present.

### Cationic Polymers

The systems according to the disclosure comprise at least one cationic polymer, other than cationic starch polymers. Optionally, the systems may, in some embodiments, comprise at least two, at least three, at least four, etc., cationic polymers other than cationic starch polymers.

As used herein, a “cationic polymer” is any polymer containing cationic groups and/or groups which can be ionized to cationic groups, and is understood to mean polymers other than cationic starch polymers. Non-limiting examples of cationic polymers that can be used include polyamine, polyaminoamide, quaternary polyammonium polymers, or mixtures thereof. Exemplary polyamine, polyaminoamide, and quaternary poly-ammonium polymers that can be used include those described in French patents FR 2 505 348 and FR 2 542 997. Such polymers can be chosen from the following:

(1) homopolymers or copolymers derived from esters or amides of acrylic or methacrylic acid;

(2) cationic cellulose derivatives such as (a) the cellulose ether derivatives containing quaternary ammonium groups that are described, for example, in French patent FR 1 492 597, or (b) the copolymers of cellulose or cellulose derivatives grafted with a water-soluble quaternary ammonium monomer, which are described, for example, in U.S. Pat. No. 4,131,576, such as hydroxyalkylcelluloses, for instance hydroxymethyl-, hydroxyethyl- or hydroxypropyl celluloses grafted with a methacryloylethyltrimethyl-ammonium, methacrylamidopropyltrimethyl-ammonium or dimethyldiallylammonium salt. A non-limiting example that may be mentioned is polyquaternium-10;

(3) other cationic polysaccharides such as those described, for example, in U.S. Pat. Nos. 3,589,578 and 4,031,307, such as guar gums containing cationic trialkyl-ammonium groups;

(4) polymers composed of piperazinyl units and divalent alkylene or hydroxyalkylene groups having straight or branched chains which are optionally interrupted by oxygen, sulphur or nitrogen atoms or by aromatic or heterocyclic rings, and also the oxidation and/or quaternization products of these polymers. Polymers of this kind are described, for example, in French patents FR 2 162 025 and FR 2 280 361;

(5) polyaminoamides which are soluble in water, such as those described in French patents FR 2 252 840 and FR 2 368 508;

(6) polyaminoamide derivatives, for example, the adipic acid/dialkylaminohydroxy-alkyldialkylenetriamine polymers in which the alkyl group contains 1 to 4 carbon atoms and is preferably a methyl, ethyl or propyl group, and the alkylene group contains 1 to 4 carbon atoms and is preferably the ethylene group. Polymers of this kind are described, for example, in French patent FR 1 583 363;

(7) polymers obtained by reacting a polyalkylene-poly-amine containing two primary amine groups and at least one secondary amine group with a dicarboxylic acid selected from diglycolic acid and saturated aliphatic dicarboxylic acids having 3 to 8 carbon atoms. The molar ratio of the polyalkylene-polyamine to the dicarboxylic acid is between 0.8:1 and 1.4:1, and the resulting polyaminoamide is reacted with epichlorohydrin in a molar ratio of epichlorohydrin to the secondary amine group of the polyaminoamide of between 0.5:1 and 1.8:1. Polymers of this kind are described, for example, in U.S. Pat. Nos. 3,227,615 and 2,961,347;

(8) alkyldiallylamine or dialkyldiallylammonium cyclopolymers such as the homopolymer of dimethyl-diallylammonium chloride, and the copolymers of diallyldimethylammonium chloride and acrylamide;

9) quaternary diammonium polymers having a number-average molecular mass of generally between 1000 and 100 000, such as those described, for example, in French patents FR 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020; an example that may be mentioned is hexadimethrine chloride (INCI name), sold by Chimex under the name Mexomere PO;

(10) quaternary polyammonium polymers such as those described, for example, in patent application EP-A-122 324;

(11) quaternary vinylpyrrolidone and vinylimidazole polymers such as, for example, the products sold under the names Luviquat® FC 905, FC 550 and FC 370 by BASF;

(12) polyamines such as Polyquart® H sold by Henkel, listed under the name “POLYETHYLENE GLYCOL (15) TALLOW POLYAMINE” in the CTFA dictionary; and

(13) crosslinked polymers of methacryloyloxyalkyl(C1-C4) trialkyl(C1-C4) ammonium salts, such as those sold under the name Salcare® SC 92, Salcare® SC 95 and Salcare® SC 96 by Allied Colloids.

In some embodiments, the cationic polymer may be chosen from Polyquaternium 5, Polyquaternium-6, Polyquaternium-7, Polyquaternium-10, Polyquaternium-15, Polyquaternium-16, Polyquaternium-17, Polyquaternium-18, Polyquaternium-22, Polyquaternium-28, Polyquaternium-32, Polyquaternium-37, Polyquaternium-39, Polyquaternium-46, Polyquaternium-47, Polyquaternium-53, Quaternium-14, Quaternium-15, Quaternium-22, Quaternium-24, Quaternium-26, Quaternium-72, or Quaternium-82.

As other examples of cationic polymers, poly(methacryloyloxyethyl trimethylammonium chloride), quaternized cellulose derivatives, cationic alkyl polyglycosides, cationized honey, cationic guar derivatives, polymeric dimethyl diallyl ammonium salts and copolymers thereof with esters and amides of acrylic acid and methacrylic acid, copolymers of vinyl pyrrolidone with quaternized derivatives of dialkylaminoalkyl acrylate and methacrylate, vinyl pyrrolidone-vinyl imidazolium methochloride copolymers, or quaternized polyvinyl alcohol may be chosen.

Mixtures of two or more of any of the above may also be used. In at least some embodiments, the cationic polymer comprises, consists essentially of, or consists of Polyquaternium-6, Polyquaternium-15, Polyquaternium-22, Polyquaternium-32, Polyquaternium-37, Polyquaternium-39, Polyquaternium-47, Quaternium-14, Quaternium-22, Quaternium-24, or mixtures of two or more thereof. In some embodiments, the cationic polymer comprises, consists essentially of, or consists of Polyquaternium-37.

The cationic polymers are present in the systems in a total amount sufficient, in combination with the other components of the systems, to stabilize the composition in which the system is included. For example, the total amount of cationic polymers can be at least about 0.35%, such as at least about 0.4%, at least about 0.45%, or at least about 0.5% by weight, relative to the total weight of the composition in which the system is present. For example, the systems may comprise a total amount of cationic polymers ranging from about 0.35% to about 5%, from about 0.4% to about 4%, from about 0.45% to about 3%, or from about 0.5% to about 2% by weight, relative to the total weight of the composition in which the system is present. In various embodiments, the system comprises a total amount of cationic polymers that is not less than 0.35%, such as not less 0.4%, not less than 0.45%, or not less than 0.5% by weight, relative to the total weight of the composition in which the system is present.

In various embodiments, the cationic polymers are present in the system in a total amount ranging from about 0.35% to about 8%, such as from about 0.35% to about 7%, from about 0.35% to about 6%, from about 0.35% to about 5%, from about 0.35% to about 4.5%, from about 0.35% to about 4%, from about 0.35% to about 3.5%, from about 0.35% to about 3%, from about 0.35% to about 2.5%, from about 0.35% to about 2%, from about 0.35% to about 1.75%, from about 0.35% to about 1.5%, from about 0.35% to about 1.25%, from about 0.35% to about 1%, from about 0.35% to about 0.75%, from about 0.4% to about 8%, from about 0.4% to about 7%, from about 0.4% to about 6%, from about 0.4% to about 5%, from about 0.4% to about 4.5%, from about 0.4% to about 4%, from about 0.4% to about 3.5%, from about 0.4% to about 3%, from about 0.4% to about 2.5%, from about 0.4% to about 2%, from about 0.4% to about 1.75%, from about 0.4% to about 1.5%, from about 0.4% to about 1.25%, from about 0.4% to about 1%, from about 0.4% to about 0.75%, from about 0.45% to about 8%, from about 0.45% to about 7%, from about 0.45% to about 6%, from about 0.45% to about 5%, from about 0.45% to about 4.5%, from about 0.45% to about 4%, from about 0.45% to about 3.5%, from about 0.45% to about 3%, from about 0.45% to about 2.5%, from about 0.45% to about 2%, from about 0.45% to about 1.75%, from about 0.45% to about 1.5%, from about 0.45% to about 1.25%, from about 0.45% to about 1%, from about 0.45% to about 0.75%, from about 0.5% to about 8%, from about 0.5% to about 7%, from about 0.5% to about 6%, from about 0.5% to about 5%, from about 0.5% to about 4.5%, from about 0.5% to about 4%, from about 0.5% to about 3.5%, from about 0.5% to about 3%, from about 0.5% to about 2.5%, from about 0.5% to about 2%, from about 0.5% to about 1.75%, from about 0.5% to about 1.5%, from about 0.5% to about 1.25%, from about 0.5% to about 1%, or from about 0.5% to about 0.75% by weight, relative to the total weight of the composition in which the system is present.

In certain embodiments, it may be particularly advantageous to choose amounts of starch polymers and cationic polymers such that the system (and the composition comprising the system) has a weight ratio of the total amount of starch polymers to the total amount of cationic polymers of greater than 1, such as greater than about 1.25, greater than about 1.5, greater than about 1.75, greater than about 2, greater than about 2.25, greater than about 2.5, greater than about 2.75, or greater than about 3. For example, the weight ratio of starch polymer(s) to cationic polymer(s) may range from greater than 1 to about 6, such as from greater than 1 to about 5, from greater than 1 to about 4, from greater than 1 to about 3.5, from greater than 1 to about 3, from greater than 1 to about 2.5, from greater than 1 to about 2.25, from greater than 1 to about 2, from greater than 1 to about 1.75, from greater than 1 to about 1.5, or from greater than 1 to about 1.25. In some embodiments, the weight ratio of starch polymer(s) to cationic polymer(s) may range from 1.1 to 5, from 1.2 to 4, from 1.3 to 3, from 1.5 to 2.5, or may range from about 1.25 to about 5, from about 1.25 to about 4, from about 1.25 to about 3, from about 1.25 to about 2.5, from about 1.5 to about 5, from about 1.5 to about 4, from about 1.5 to about 3.5, from about 1.5 to about 3, from about 1.5 to about 2.5, from about 1.5 to about 2.25, or from about 1.5 to about 2.

In further embodiments, the weight ratio of starch polymer(s) to cationic polymer(s) may range from about 1.25 to about 8, such as from about 1.5 to about 8, from about 1.75 to about 8, from about 2 to about 8, from about 2.25 to about 8, from about 2.5 to about 8, from about 2.75 to about 8, from about 3 to about 8, from about 1.25 to about 7, from about 1.5 to about 7, from about 1.75 to about 7, from about 2 to about 7, from about 2.25 to about 7, from about 2.5 to about 7, from about 2.75 to about 7, from about 3 to about 7, from about 1.25 to about 6, from about 1.5 to about 6, from about 1.75 to about 6, from about 2 to about 6, from about 2.25 to about 6, from about 2.5 to about 6, from about 2.75 to about 6, from about 3 to about 6, from about 1.25 to about 5, from about 1.5 to about 5, from about 1.75 to about 5, from about 2 to about 5, from about 2.25 to about 5, from about 2.5 to about 5, from about 2.75 to about 5, or from about 3 to about 5.

### Fatty Alcohols

Systems according to the disclosure comprise at least one fatty alcohol. The systems may, in some embodiments, comprise at least two, at least three, at least four, etc., fatty alcohols.

The fatty alcohols that are useful in the systems include alcohols comprising at least one hydroxyl group (OH), and comprising at least 8 carbon atoms, and which are neither oxyalkylenated (in particular neither oxyethylenated nor oxypropylenated) nor glycerolated. The fatty alcohols can be represented by the general formula R—OH, wherein R denotes a saturated (alkyl) or unsaturated (alkenyl) group, linear or branched, comprising from 8 to 40 carbon atoms, for example from 10 to 30 carbon atoms, from 12 to 24 carbon atoms, or from 14 to 22 carbon atoms. The fatty alcohol(s) may be liquid or solid, and the systems may comprise one or more liquid fatty alcohols, one or more solid fatty alcohols, or a mixture of one or more liquid fatty alcohols and one or more solid fatty alcohols.

In some embodiments, the systems comprise at least one solid fatty alcohol. The solid fatty alcohols that can be used include those that are solid at ambient temperature and at atmospheric pressure (25° C., 780 mmHg), and are insoluble in water, that is to say they have a water solubility of less than 1% by weight, preferably less than 0.5% by weight, at 25° C., 1 atm.

In some embodiments, the systems comprise at least one liquid fatty alcohol. The liquid fatty alcohols, such as those containing 10 to 34 carbon atoms, may in some instances have branched carbon chains and/or have one or more double bonds, for example from 1 to 3 double bonds. Thus, in some embodiments, the fatty alcohol is chosen from branched and/or unsaturated liquid fatty alcohols containing from 12 to 40 carbon atoms. In some embodiments, the liquid fatty alcohol is chosen from branched saturated alcohols.

Non-limiting examples of useful fatty alcohols include lauryl alcohol (1-dodecanol), myristic or myristyl alcohol (1-tetradecanol), cetyl alcohol (1-hexadecanol), stearyl alcohol (1-octadecanol), cetearyl alcohol, arachidyl alcohol (1-eicosanol), behenyl alcohol (1-docosanol), lignoceryl alcohol (1-tetracosanol), ceryl alcohol (1-hexacosanol), montanyl alcohol (1-octacosanol), myricylic alcohol (1-triacontanol), oleic alcohol, linoleic alcohol, linolenic alcohol, isocetyl alcohol, isostearyl alcohol, octyldodecanol (2-octyl-1-dodecanol), 2-butyloctanol, 2-hexyl-1-decanol, 2-decyl-1-tetradecanol, 2-tetradecyl-1-cetanol, or mixtures thereof.

In certain embodiments, the one or more fatty alcohols are chosen from those having from 12 to 24 carbon atoms, for example octyldodecanol, cetyl alcohol, stearyl alcohol, cetearyl alcohol, behenyl alcohol, lauryl alcohol, myristic or myristyl alcohol, arachidyl alcohol, lignoceryl alcohol, or mixtures thereof. In some embodiments, the fatty alcohol comprises, consists essentially of, or consists of cetyl alcohol, stearyl alcohol, cetearyl alcohol, octyldodecanol, or mixtures of two or more thereof.

The fatty alcohols are present in the systems in a total amount sufficient, in combination with the other components of the systems, to stabilize the composition in which the system is included. For example, the fatty alcohols can be present in an amount ranging from about 0.5% to about 10%, such as from about 1% to about 10%, from about 1% to about 8%, from about 1.5% to about 7%, from about 2% to about 6%, or from about 3% to about 5% by weight, relative to the total weight of the composition in which the system is present.

By way of non-limiting example, the total amount of fatty alcohols may range from about 0.5% to about 10%, about 0.5% to about 9%, about 0.5% to about 8%, about 0.5% to about 7%, about 0.5% to about 6%, about 0.5% to about 5%, about 0.5% to about 4%, about 0.5% to about 3%, about 0.5% to about 2%, about 0.5% to about 1%, about 1% to about 10%, about 1% to about 9%, about 1% to about 8%, about 1% to about 7%, about 1% to about 6%, about 1% to about 5%, about 1% to about 4%, about 1% to about 3%, about 1% to about 2%, about 1.5% to about 10%, about 1.5% to about 9%, about 1.5% to about 8%, about 1.5% to about 7%, about 1.5% to about 6%, about 1.5% to about 5%, about 1.5% to about 4%, about 1.5% to about 3%, about 1.5% to about 2%, about 2% to about 10%, about 2% to about 9%, about 2% to about 8%, about 2% to about 7%, about 2% to about 6%, about 2% to about 5%, about 2% to about 4%, about 2% to about 3%, about 2.5% to about 10%, about 2.5% to about 9%, about 2.5% to about 8%, about 2.5% to about 7%, about 2.5% to about 6%, about 2.5% to about 5%, about 2.5% to about 4%, about 2.5% to about 3%, about 3% to about 10%, about 3% to about 9%, about 3% to about 8%, about 3% to about 7%, about 3% to about 6%, about 3% to about 5%, or about 3% to about 4% by weight, relative to the total weight of the composition in which the system is present.

### II. Compositions

Compositions according to the disclosure include the systems described herein. Compositions comprising at least one electrolyte, which tend to be unstable, may particularly benefit from use of the systems described herein. Exemplary compositions that can be stabilized by the systems comprise at least one electrolyte in addition to various components typically used in compositions for treating keratinous substrates, caring for keratinous substrates, conditioning keratinous substrates, etc. Although exemplary components and compositions are provided herein, it should be understood that other compositions can also be stabilized using the systems described herein, and other components may be chosen.

### Electrolytes

Compositions that can be stabilized with systems according to the disclosure generally comprise at least one electrolyte. Compositions comprising electrolytes, particularly at levels above about 0.1%, can be used, for example, to prevent damage to keratinous substrates, repair damaged keratinous substrates, or provide strength to keratinous substrates, but such compositions are known to be unstable.

As used herein, the term “electrolyte” means compounds such as salts, acids, and bases that form ions when present in a composition comprising a polar solvent, such as water. Although not limiting, the electrolytes may include an anion such as phosphate, chloride, sulfate, or citrate, and a cation such as sodium, ammonium, potassium, or magnesium. It can refer to a compound that is added to the composition (e.g. sodium citrate) or a compound that may be formed in situ via balancing counterions from other components.

The electrolytes may therefore, in some embodiments, be added to the composition as an acid or a salt. By way of example, acids that can be used include non-polymeric, mono, di, and/or tri-carboxylic acids, such as formic acid, acetic acid, propionic acid, butyric acid, valeric acid, caproic acid, entanthic acid, caprylic acid, pelargonic acid, capric acid, undecylic acid, lauric acid, tridecylic acid, lauric acid, tridecylic acid, myristic acid, pentadecylic acid, palmitic acid, margaric acid, stearic acid, nonadecylic acid, arachidic acid, lactic acid, oxalic acid, malonic acid, malic acid, glutaric acid, citraconic acid, succinic acid, adipic acid, tartaric acid, fumaric acid, maleic acid, sebacic acid, azelaic acid, dodecanedioic acid, phthalic acid, isophthalic acid, terephthalic acid, 2,6-naphthalene dicarboxylic acid, citric acid, isocitric acid, aconitric acid, propane-1,2,3-tricarboxylic acid, and benzene-1,3,5-tricarboxylic acid.

Salts of any of the above-mentioned acids may also be used, for example the sodium, ammonium, potassium, and/or magnesium salts of the acids. By way of non-limiting example only, the salt may be sodium citrate, sodium chloride, sodium sulfate, ammonium sulfate, ammonium chloride, potassium citrate, potassium chloride, etc.

The amount of electrolyte in the composition can vary, but is typically greater than about 0.1%, such as greater than about 0.25%, greater than about 0.5%, greater than about 0.75%, or greater than about 1% by weight, relative to the total weight of the composition. In some embodiments, the total amount of electrolytes may range from about 0.5% to about 5%, from about 0.5% to about 4%, from about 0.5% to about 3%, from about 0.5% to about 2%, from about 0.5% to about 1.5%, from about 0.6% to about 5%, from about 0.6% to about 4%, from about 0.6% to about 3%, from about 0.6% to about 2%, from about 0.6% to about 1.5%, from about 0.7% to about 5%, from about 0.7% to about 4%, from about 0.7% to about 3%, from about 0.7% to about 2%, from about 0.7% to about 1.5%, from about 0.8% to about 5%, from about 0.8% to about 4%, from about 0.8% to about 3%, from about 0.8% to about 2%, from about 0.8% to about 1.5%, from about 0.9% to about 5%, from about 0.9% to about 4%, from about 0.9% to about 3%, from about 0.9% to about 2%, or from about 0.9% to about 1.5% by weight, relative to the total weight of the composition.

### Surfactants

Compositions for treating keratin substrates, caring for keratin substrates, conditioning keratin substrates, etc., often comprise one or more surfactants. Surfactants may be anionic, cationic, nonionic, or amphoteric, and compositions may comprise mixtures of surfactants having the same or different ionicities.

For example, anionic surfactants that may be chosen include alkyl sulfates and alkyl ether sulfates, such as, for example, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium trideceth-1 sulfate, sulfate, sodium trideceth-2 sulfate, sulfate, sodium trideceth-3 sulfate, sodium tridecyl sulfate, sodium methyl lauroyl taurate, sodium undecyl sulfate, sodium decyl sulfate, sodium methyl cocoyl taurate, sodium lauroyl isethionate, sodium cocoyl isethionate, sodium laurethsulfosuccinate, sodium laurylsulfosuccinate, sodium tridecyl benzene sulfonate, or sodium dodecyl benzene sulfonate.

Typical amphoteric surfactants include derivatives of aliphatic secondary and tertiary amines where the aliphatic radical can be straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, and amidobetaines and amidosulfobetaines, oleyl betaine, or cocamidopropyl betaine may be chosen.

Nonionic surfactants may include, for example, polyoxyethylene glyceryl fatty acid esters, sorbitan fatty acid esters, polyoxyethylene sorbitan fatty acid esters, polyoxyethylene alkyl ethers, alkylated polysaccharides, alkyl glycosides, sucrose fatty acid esters, polyglycol fatty acid esters, alkaloyl diethanolamides, and fatty acid isopropanolamides. For example, ethoxylated or propoxylated alcohols and alkyl phenols, which, in various embodiments, contain a straight or branched C8-22 alkyl group, for example C10-20 or C12-20, and from about 6 to about 30, such as from about 6 to about 25, ethylene oxide or propylene oxide groups (high HLB nonionic surfactants) or from about 1 to about 5 ethylene oxide or propylene oxide groups (low HLB nonionic surfactants), or esters of acids and alcohols, e.g. glyceryl esters of fatty acids, may be present. As examples, nonionic surfactants may be chosen from stearyl alcohol polyoxyethylenated with 2 EO (Steareth-2), glyceryl laurate, glyceryl stearate, glycol stearate, sorbitan isostearate; sorbitan oleate; sorbitan sesquioleate, sorbitan stearate, sorbitan trioleate, cetyl alcohol polyoxyethylenated with 20 EO (Ceteth-20), cetyl alcohol polyoxyethylenated with 24 EO (Ceteth-24), lauryl alcohol polyoxyethylenated with 21 EO (Laureth-21), lauryl alcohol polyoxyethylenated with 23 EO (Laureth-23), stearyl alcohol polyoxyethylenated with 20 EO (Steareth-20), stearyl alcohol polyoxyethylenated with 21 EO (Steareth-21), polyoxyethylene (20) sorbitan monolaurate (Polysorbate-20), or polyoxyethylene (6) oleyl ether (Oleth-20).

Cationic surfactants that may be chosen include quaternary alkyl amines such as those of the general formula (I) below:

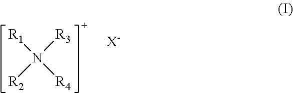

wherein R₁, R₂, R₃ and R₄ are independently chosen from saturated or unsaturated, linear or branched, aliphatic hydrocarbon radicals comprising from 1 to about 30 carbon atoms, or an alkoxy, alkoxycarbonylalkyl, polyoxyalkylene, alkylamido, alkylamidoalkyl, hydroxyalkyl, aromatic, aryl, or alkylaryl radical comprising from about 12 to about 30 carbon atoms, and X⁻ is an anion, for example a halide, e.g., chloride, bromide, or iodide, a (C1-C4) alkyl sulfate, a (C1-C4) alkylsulfonate, or a (C1-C4) alkylarylsulfonate, a methanesulfonate, a phosphate, a nitrate, a tosylate, an anion derived from an organic acid such as an acetate or a lactate, or any other anion that is compatible with the ammonium bearing an ester function. In some embodiments, the anion X⁻ is bromide, chloride, or methosulfate. Optionally, at least one of R₁, R₂, R₃ and R₄ has at least 8 carbon atoms, such as from 12 to 30 carbon atoms, from 16 to 24 carbon atoms, or from 18 to 22 carbon atoms.

In some compositions, the quaternary alkyl amines may be chosen from monoalkyl quaternary alkyl amines, dialkyl quaternary alkyl amines, salts thereof, or mixtures thereof, such as behenyltrimethylammonium bromide, behenyltrimethylammonium chloride, behenyltrimethylammonium methosulfate, distearyldimethylammonium bromide, distearyldimethylammonium chloride, distearyldimethylammonium methosulfate, cetyltrimethylammonium bromide, cetyltrimethylammonium chloride, cetyltrimethylammonium methosulfate, dodecyltrimethylammonium bromide, dodecyltrimethylammonium chloride, or dodecyltrimethylammonium methosulfate.

As further examples of cationic surfactants, steartrimonium chloride, stearalkonium chloride, dicetyldimonium chloride, hydroxypropyltrimonium chloride, cocotrimonium methosulfate, olealkonium chloride, steartrimonium chloride, babassuamidopropalkonium chloride, brassicamidopropyl dimethylamine, behenalkonium chloride, benzethonium chloride, cetylpyridinium chloride, lauralkonium chloride, cetalkonium chloride, cethylamine hydrofluoride, or mixtures thereof may be used.

Amido amines may also be chosen. For example, the compositions may comprise tertiary amido amines having an alkyl group of from about 12 to about 22 carbons, such as brassicamidopropyl dimethylamine, stearamidopropyldimethylamine, stearamidopropyldiethylamine, stearamidoethyldiethylamine, stearamidoethyldimethyl-amine, palmitamidopropyldimethylamine, palmitamidopropyldiethylamine, palmitamido-ethyldiethylamine, palmitamidoethyldimethylamine, behenamidopropyldimethylamine, behenamidopropyldiethylamine, behenamidoethyldiethylamine, behenamidoethyl-dimethylamine, arachidamidopropyldimethylamine, arachidamidopropyldiethylamine, arachidamidoethyldiethylamine, arachidamidoethyldimethylamine, or diethylaminoethyl-stearamide.

Typically, the total amount of surfactants may vary, but can range up to about 15%, such as from about 0.5% to about 12%, from about 1% to about 10%, from about 1.5% to about 8%, or from about 2% to about 5% by weight, relative to the total weight of the composition. In certain embodiments, the total amount of surfactants ranges from about 0.5% to about 15%, such as from about 1% to about 15%, from about 1% to about 12%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1.5% to about 15%, from about 1.5% to about 12%, from about 1.5% to about 10%, from about 1.5% to about 9%, from about 1.5% to about 8%, from about 1.5% to about 7%, from about 1.5% to about 6%, from about 1.5% to about 5%, from about 1.5% to about 4%, from about 1.5% to about 3%, from about 2% to about 15%, from about 2% to about 12%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 2.5% to about 15%, from about 2.5% to about 12%, from about 2.5% to about 10%, from about 2.5% to about 9%, from about 2.5% to about 8%, from about 2.5% to about 7%, from about 2.5% to about 6%, from about 2.5% to about 5%, from about 2.5% to about 4%, from about 3% to about 15%, from about 3% to about 12%, from about 3% to about 10%, from about 3% to about 9%, from about 3% to about 8%, from about 3% to about 7%, from about 3% to about 6%, from about 3% to about 5%, from about 3% to about 4%, from about 3.5% to about 15%, from about 3.5% to about 12%, from about 3.5% to about 10%, from about 3.5% to about 9%, from about 3.5% to about 8%, from about 3.5% to about 7%, from about 3.5% to about 6%, from about 3.5% to about 5%, or from about 3.5% to about 4% by weight, relative to the total weight of the composition.

### Solvent(s)

Compositions for treating keratin substrates, caring for keratin substrates, conditioning keratin substrates, etc., generally comprise one or more solvents, for example water and/or at least one non-aqueous solvent. The compositions may comprise water in an amount ranging from about 30% to about 95%, such as from about 50% to about 95%, about 60% to about 95%, about 70% to about 95%, about 75% to about 95%, about 80% to about 95%, about 60% to about 90%, about 65% to about 90%, about 70% to about 90%, about 75% to about 90%, or about 80% to about 90% by weight, based on the total weight of the composition.

In some embodiments, the solvent includes one or more non-aqueous solvents, for example, glycerin, C1-4 alcohols, organic solvents, fatty alcohols, fatty ethers, fatty esters, polyols, glycols, vegetable oils, mineral oils, liposomes, laminar lipid materials, or any a mixture thereof. Non-limiting examples of non-aqueous solvents which may be used include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether, or mixtures of two or more thereof.

### Additional Fatty Compounds

Compositions for treating keratin substrates, caring for keratin substrates, conditioning keratin substrates, etc., often comprise one or more fatty compounds other than the fatty alcohols as described above.

For example, the compositions may optionally comprise oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), optionally branched and/or unsaturated fatty acids (e.g. myristic acid, palmitic acid, stearic acid, behenic acid, oleic acid, linoleic acid, linolenic acid, or isostearic acid), fatty alcohols other than those described above (e.g. alkoxylated fatty alcohols), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C5-C9 esters, trimethylolpropane polyesters, propylene glycol polyesters, or polyesters of hydrogenated castor oil), perfluorinated and/or organofluorinated oils, volatile or non-volatile silicone oils, fluorosilicone oils, or mixtures of two or more thereof. In some embodiments, the composition may be free or substantially free of silicone oils.

If present, the total amount of additional fatty compounds may range up to about 30%, such as from about 0.1% to about 20%, from about 0.5% to about 15%, from about 1% to about 10%, from about 1.5% to about 8%, or from about 2% to about 5% by weight, relative to the total weight of the composition. For example, the total amount of additional fatty compounds may range from about 1% to about 15%, from about 1% to about 12%, from about 1% to about 7%, from about 1% to about 5%, from about 1% to about 4%, from about 1% to about 3%, from about 1.5% to about 15%, from about 1.5% to about 12%, from about 1.5% to about 10%, from about 1.5% to about 7%, from about 1.5% to about 5%, from about 1.5% to about 4%, from about 1.5% to about 3%, from about 2% to about 15%, from about 2% to about 12%, from about 2% to about 10%, from about 2% to about 7%, from about 2% to about 5%, from about 2% to about 4%, from about 2% to about 3%, from about 2.5% to about 15%, from about 2.5% to about 12%, from about 2.5% to about 10%, from about 2.5% to about 7%, from about 2.5% to about 5%, from about 2.5% to about 4%, or from about 2.5% to about 3.5% by weight, relative to the total weight of the composition.

### Thickening Agents

Compositions for treating keratin substrates, caring for keratin substrates, conditioning keratin substrates, etc., often comprise one or more thickening agents other than starch polymers.

Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, celluloses such as hydroxyethylcellulose and hydroxypropylcellulose, and guars such as hydroxypropyl guar.

In some embodiments, the thickening agent may be chosen from associative thickening polymers such as anionic associative polymers, amphoteric associative polymers, cationic associative polymers, or nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may vary, but may be, for example, at least about 0.1%, such as at least about 0.25%, at least about 0.5%, at least about 0.75%, or at least about 1% by weight, based on the total weight of the composition. In some embodiments, the total amount of thickening agents may range from about 0.5% to about 5%, from about 0.5% to about 4%, from about 0.5% to about 3%, from about 0.5% to about 2%, from about 0.5% to about 1.5%, from about 0.6% to about 5%, from about 0.6% to about 4%, from about 0.6% to about 3%, from about 0.6% to about 2%, from about 0.6% to about 1.5%, from about 0.7% to about 5%, from about 0.7% to about 4%, from about 0.7% to about 3%, from about 0.7% to about 2%, from about 0.7% to about 1.5%, from about 0.8% to about 5%, from about 0.8% to about 4%, from about 0.8% to about 3%, from about 0.8% to about 2%, from about 0.8% to about 1.5%, from about 0.9% to about 5%, from about 0.9% to about 4%, from about 0.9% to about 3%, from about 0.9% to about 2%, or from about 0.9% to about 1.5% by weight, relative to the total weight of the composition.

### Adjuvants

Typically, compositions for treating keratin substrates, caring for keratin substrates, conditioning keratin substrates, etc., comprise one or more adjuvants. Non-limiting examples include preservatives, fragrances, pH adjusters, salts, antioxidants, vitamins, vitamin derivatives, botanical extracts, UV filtering agents, proteins, protein hydrolysates, protein isolates, hydrotropes, pearlescent agents, buffers, colorants for coloring the compositions (not for coloring the hair), sequestering agents, and the like.

The total amount of adjuvants, if present, typically ranges from about 0.01% to about 10% based on the total weight of the composition. For example, in some embodiments the individual amounts of each adjuvant or the total amount of adjuvants may range from about 0.1% to about 10%, about 0.1% to about 8%, about 0.1% to about 5%, about 0.1% to about 4%, about 0.1% to about 3%, about 0.1% to about 2%, about 1% to about 10%, about 1% to about 8%, about 1% to about 5%, about 1% to about 4%, about 1% to about 3%, or about 1% to about 2% by weight, based on the total weight of the composition.

Compositions comprising the systems according to the disclosure generally have a viscosity of less than about 30000 mPas, such as less than about 25000 mPas, less than about 20000 mPas, less than about 18000 mPas, less than about 15000 mPas, less than about 12000 mPas, less than about 10000 mPas, or less than about 8000 mPas, when measured with a Brookfield viscometer, spindle RV-05, at 5 RPM for one minute under ambient conditions. Surprisingly, the viscosity of the composition comprising the systems according to the disclosure stays relatively stable for a period of at least about 4 weeks, and in some embodiments at least about 8 weeks, at least about 12 weeks, at least about 24 weeks, at least about 6 months, at least about 12 months, at least about 2 years, at least about 3 years, or more.

Compositions comprising systems according to the disclosure are surprisingly stable in that no phase separation is observed (visually) in the stabilized compositions over a period of time. For example, by including the systems according to the disclosure in various compositions for keratin substrates that comprise at least one electrolyte, no phase separation is observed for at least about 4 weeks, and in some embodiments at least about 8 weeks, at least about 12 weeks, at least about 24 weeks, at least about 6 months, at least about 12 months, at least about 2 years, at least about 3 years, or more.

As such, the disclosure also relates to methods of stabilizing compositions, in particular compositions comprising at least one electrolyte, by including or employing a system according to the disclosure in the compositions. The methods for including or employing systems according to the disclosure in the compositions to be stabilized can be by any means known to those of skill in the art for preparing such compositions, as long as the components of the systems described herein are incorporated into the composition in total combined amounts sufficient to stabilize the composition. No specific steps are required in order to prepare such stable compositions.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “citric acid and/or salts thereof” means “citric acid and salts thereof” as well as “citric acid or salts thereof,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts herein are given based upon the total weight of the composition comprising a system according to the disclosure, unless otherwise indicated. Unless otherwise indicated, all percentages are by weight of active material. It is to be understood that the language “based on the total weight of the composition,” “based on the weight of the composition in which the system is present,” “relative to the total weight of the composition,” and the like, refers to the weight of the composition including the components of the system as well as any components present in the composition other than those described for the systems described herein.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

It is to be understood that, with regard to salts of carboxylic acids described herein, it is intended to encompass the use of a salt of a carboxylic acid as an ingredient added to a composition according to the disclosure, or to the ion or salt of the carboxylic acid that forms when the carboxylic acid is used as an ingredient in a composition according to the disclosure.

As used herein, the term “substantially free” or “essentially free” means the specific material may be present in small amounts that do not materially affect the basic and novel characteristics of the compositions according to the disclosure. For instance, there may be less than 2% by weight of a specific material added to a composition, based on the total weight of the compositions (provided that an amount of less than 2% by weight does not materially affect the basic and novel characteristics of the compositions according to the disclosure. Similarly, the compositions may include less than 2%, less than 1.5%, less than 1%, less than 0.5%, less than 0.1%, less than 0.05%, or less than 0.01%, or none of the specified material. Furthermore, all components that are positively set forth in the instant disclosure may be negatively excluded from the claims, e.g., a claimed composition may be “free,” “essentially free” (or “substantially free”) of one or more components that are positively set forth in the instant disclosure. The term “substantially free” or “essentially free” as used herein may also mean that the specific material is not added to the composition but may still be present in a raw material that is included in the composition.

For purposes of the present disclosure, the term “system” or “stabilizing system” means the combination of (a) at least one starch polymer, (b) at least one cationic polymer, and (c) at least one fatty alcohol as described herein, wherein the components are present in a composition in combined amounts sufficient to stabilize the composition.

Unless otherwise expressly stated, it is in no way intended that any method set forth herein be construed as requiring that its steps be performed in a specific order.

### EXAMPLES

The following Examples are intended to be non-limiting and explanatory in nature only. In the Examples, amounts are expressed in percentage by weight (wt %) of active materials, relative to the total weight of the composition, unless otherwise indicated.

### Example 1—Stability Study

The stability of compositions comprising systems according to the disclosure, and compositions not comprising systems according to the disclosure, was evaluated. The compositions in Table 1, including composition 1A that employs a system according to the disclosure and comparative compositions C1-C6, were prepared. Each of compositions 1A and C1-C6 can be used for treating, caring for, and/or conditioning keratin fibers, such as hair.

TABLE 1

| | Inv. | Comparative | | | | | |
|---|---|---|---|---|---|---|---|
| | 1A | C1 | C2 | C3 | C4 | C5 | C6 |
| POTATO STARCH | 1.00 | 0.50 | 0.50 | 1.00 | — | 1.00 | 1.00 |
| (MODIFIED) | | | | | | | |
| POLYQUATERNIUM-37 | 0.50 | 0.25 | 0.50 | — | 0.50 | — | 0.50 |
| POLYQUATERNIUM-4 | — | — | — | — | — | 0.50 | — |
| CETEARYL ALCOHOL | 3.00 | 3.00 | 3.00 | 3.00 | 3.00 | 3.00 | — |
| OCTYLDODECANOL | 1.00 | 1.00 | 1.00 | 1.00 | 1.00 | 1.00 | — |
| SODIUM CITRATE | 1.15 | 1.15 | 1.15 | 1.15 | 1.15 | 1.15 | 1.15 |
| CITRIC ACID | 1.00 | 1.00 | 1.00 | 1.00 | 1.00 | 1.00 | 1.00 |
| NONIONIC SURFACTANTS | 0.99 | 0.95 | 0.95 | 0.95 | 0.95 | 0.95 | 0.95 |
| (sorbitan oleate and/or | | | | | | | |
| glyceryl stearate and/or | | | | | | | |
| Polysorbate 20) | | | | | | | |
| CATIONIC SURFACTANTS | 5.0 | 5.0 | 5.0 | 5.0 | 5.0 | 5.0 | 5.0 |
| (behentrimonium chloride | | | | | | | |
| and/or stearamidopropyl | | | | | | | |
| dimethylamine) | | | | | | | |
| PROPYLENE GLYCOL | 0.70 | 0.70 | 0.70 | 0.70 | 0.70 | 0.70 | 0.70 |
| DICAPRYLATE/DICAPRATE | | | | | | | |
| PPG-1 TRIDECETH-6 | 0.16 | 0.16 | 0.16 | 0.16 | 0.16 | 0.16 | 0.16 |
| ADDITIVES (vitamins, pH | <2 | <2 | <2 | <2 | <2 | <2 | <2 |
| adjusters, preservatives, | | | | | | | |
| fragrance) | | | | | | | |
| SOLVENTS (water and non- | QS | QS | QS | QS | QS | QS | QS |
| aqueous solvents) | | | | | | | |

The stability of each of compositions 1A and C1-C6 was studied by subjecting the compositions to a freeze-thaw process, which is a generally accepted method for conducting an accelerated stability study. Equal amounts of each composition was placed into separate 4-ounce glass jars before being placed into the freeze-thaw chamber. The samples were then subjected to seven (7) consecutive freeze-thaw cycles, with each cycle lasting for 24 hours and including a freeze stage where the sample was held at a temperature of −10° C. for 12 hours, followed by a thaw stage where the sample was held at a temperature of 25° C. for 12 hours.

The samples were each evaluated (visually) after four (4) and seven (7) cycles. The results of the stability study are shown in Table 2, where an “X” indicates that phase separation was observed after the specified cycle, and an “√” indicates no phase separation was observed after the specified cycle.

TABLE 2

| | 1A | C1 | C2 | C3 | C4 | C5 | C6 |
|---|---|---|---|---|---|---|---|
| After 4 cycles | ✓ | X | X | ✓ | ✓ | X | X |
| After 7 cycles | ✓ | X | X | X | X | X | X |

Example 1 thus demonstrates that employing systems according to the disclosure in compositions that have at least one electrolyte surprisingly provides stability to the compositions, compared to compositions that include some of the components but not the synergistic combination of starch polymers, cationic polymers, and fatty alcohols, and/or include these components but in amounts that are insufficient to stabilize the composition.

### Example 2—Viscosity Study

The viscosity of composition 1A from Table 1, stabilized with a system according to the disclosure, was evaluated. The initial viscosity was measured within 24 hours of preparation using a Brookfield viscometer, spindle RV-05, 5 RPM for one minute under ambient conditions, and again after storage at 45° C. for 2 months. The results are shown in Table 3.

TABLE 3

| | Initial Viscosity | Viscosity After 2 Months |
|---|---|---|
| Composition 1A | 7760 mPas | 10080 mPas |

The viscosity study demonstrates that the stability imparted to compositions with systems according to the disclosure is a lasting benefit. A typical composition for treating keratinous substrates, caring for keratinous substrates, conditioning keratinous substrates, etc., would be expected to experience a significant increase in viscosity when subjected to the extreme storage conditions used in this study. However, composition 1A experienced less than a 30% increase in viscosity, which was surprising. These results indicate that the same composition, when stored under normal usage conditions, would remain stable for up to three years or more.

Thus, Example 2 demonstrates that including systems according to the disclosure in compositions with at least one electrolyte surprisingly provides long-lasting stability to such compositions.

### Example 3—Additional Compositions

The following compositions comprising systems according to the disclosure can be prepared, and are expected to have stability similar to that of composition 1A.

TABLE 4

| | 1B | 1C | 1D | 1E |
|---|---|---|---|---|
| POTATO STARCH (MODIFIED) | 2.00 | 3.50 | — | — |
| CORN STARCH | — | — | 1.00 | 2.00 |
| POLYQUATERNIUM-37 | 1.00 | 1.50 | — | — |
| POLYQUATERNIUM-32 | — | — | 0.50 | 1.50 |
| CETEARYL ALCOHOL | 5.00 | 6.00 | — | — |
| STEARYL ALCOHOL | — | — | 3.50 | 5.00 |
| SODIUM CITRATE | 0.50 | 1.00 | — | — |
| SODIUM CHLORIDE | — | — | 1.00 | 1.25 |
| CITRIC ACID | 0.50 | 0.50 | — | — |
| MALEIC ACID | — | — | 0.25 | 0.25 |
| NONIONIC SURFACTANTS | 0.99 | 0.95 | 0.95 | 0.95 |
| (sorbitan oleate and/or glyceryl | | | | |
| stearate and/or Polysorbate 20) | | | | |
| CATIONIC SURFACTANTS | 5.0 | 5.0 | 5.0 | 5.0 |
| (behentrimonium chloride and/or | | | | |
| stearamidopropyl dimethylamine) | | | | |
| PROPYLENE GLYCOL | 0.70 | 0.70 | 0.70 | 0.70 |
| DICAPRYLATE/DICAPRATE | | | | |
| PPG-1 TRIDECETH-6 | 0.16 | 0.16 | 0.16 | 0.16 |
| ADDITIVES (vitamins, pH adjusters, | <2 | <2 | <2 | <2 |
| preservatives, fragrance, etc.) | | | | |
| SOLVENTS | QS | QS | QS | QS |
| (water and non-aqueous solvents) | | | | |

1. A system for stabilizing a composition comprising at least 0.25% of electrolytes, the system comprising:
(a) at least one starch polymer;
(b) at least one cationic polymer; and
(c) at least one fatty alcohol,
wherein the total amount of starch polymers is at least about 0.75%,
wherein the total amount of cationic polymers is at least about 0.35%, and
wherein all amounts are by weight, based on the weight of the composition comprising the system.

2. The system according to claim 1, wherein the cationic polymer comprises Polyquaternium-37.

3. The system according to claim 1, wherein the total amount of starch polymers is at least about 1% by weight, based on the weight of the composition comprising the system.

4. The system according to claim 1, wherein the total amount of cationic polymers is at least about 0.5% by weight, based on the weight of the composition comprising the system.

5. The system according to claim 1, wherein the weight ratio of the total amount of starch polymers to the total amount of cationic polymers is greater than 1.

6. The system according to claim 1, wherein the total amount of fatty alcohols ranges from about 0.5% to about 10% by weight, based on the weight of the composition comprising the system.

7. A stable composition comprising:
(a) a system for stabilizing the composition comprising:
i. at least one starch polymer;
ii. at least one cationic polymer; and
iii. at least one fatty alcohol; and
(b) at least one electrolyte,
wherein the total amount of electrolytes is at least about 0.25% by weight, based on the total weight of the composition,
wherein the total amount of starch polymers is at least about 0.75% by weight, based on the total weight of the composition,
wherein the total amount of cationic polymers is at least about 0.35% by weight, based on the total weight of the composition,
wherein the total amount of fatty alcohols is at least about 0.5% by weight, based on the total weight of the composition, and
wherein the total amounts of starch polymers, cationic polymers, and fatty alcohols present in the composition stabilize the composition.

8. The stable composition according to claim 7, wherein the cationic polymer comprises Polyquaterium-37.

9. The stable composition according to claim 7, wherein the total amount of starch polymers ranges from about 0.75% to about 10% by weight, based on the weight of the composition.

10. The stable composition according to claim 7, wherein the total amount of cationic polymers ranges from about 0.35% to about 8% by weight, based on the weight of the composition.

11. The stable composition according to claim 7, wherein the weight ratio of the total amount of starch polymers to the total amount of cationic polymers is greater than 1.

12. The stable composition according to claim 7, wherein the total amount of fatty alcohols ranges from about 0.5% to about 10% by weight, based on the weight of the composition.

13. The stable composition according to claim 7, further comprising at least one surfactant chosen from nonionic surfactants, cationic surfactants, anionic surfactants, amphoteric surfactants, and mixtures thereof, where the total amount of surfactant(s) present in the composition ranges from about 0.5% to about 15% by weight, based on the weight of the composition.

14. The stable composition according to claim 7, further comprising at least one fatty compound chosen from oils of animal, vegetable, or mineral origin; linear or branched hydrocarbons; fatty acids; alkoxylated fatty alcohols; mono- and/or polyesters of fatty acids and/or of fatty alcohols; or combinations thereof.

15. The stable composition according to claim 7, having a viscosity of less than about 15000 mPas.

16. The stable composition according to claim 7, comprising:
(a) a system comprising:
i. at least one starch polymer;
ii. at least one cationic polymer; and
iii. at least one fatty alcohol;
(b) at least one electrolyte;
(c) at least one nonionic and/or cationic surfactant; and
(d) water,
wherein the total amount of electrolytes is at least about 0.5%,
wherein the total amount of starch polymers is at least about 1%,
wherein the total amount of cationic polymers is at least about 0.5%, and
wherein all amounts are by weight, based on the weight of the composition.

17. The stable composition according to claim 16, wherein the cationic polymer comprises Polyquaterium-37.

18. The stable composition according to claim 17, wherein the weight ratio of the total amount of starch polymers to the total amount of cationic polymers is greater than 1.

19. The stable composition according to claim 18, wherein:
the starch polymer comprises modified and/or unmodified potato starch, and/or
the fatty alcohol comprises octyldodecanol, cetearyl alcohol, cetyl alcohol, stearyl alcohol, or a mixture of two or more thereof.

20. A method for stabilizing a composition comprising at least one electrolyte, the method comprising including a stabilizing system in the composition, wherein the system comprises:
(a) at least one starch polymer;
(b) at least one cationic polymer; and
(c) at least one fatty alcohol,
wherein the total amount of starch polymers is at least about 0.75% by weight, based on the total weight of the composition,
wherein the total amount of cationic polymers is at least about 0.35% by weight, based on the total weight of the composition,
wherein the total amount of fatty alcohols is at least about 0.5% by weight, based on the total weight of the composition, and
wherein the total amounts of starch polymers, cationic polymers, and fatty alcohols present in the composition stabilize the composition.

21. The system according to claim 1, wherein:
the total amount of electrolytes ranges from about 0.5% to about 3% by weight, based on the total weight of the composition,
the total amount of starch polymers ranges from about 0.75% to about 4% by weight, based on the total weight of the composition,
the total amount of cationic polymers ranges from about 0.35% to about 2% by weight, based on the total weight of the composition,
the total amount of fatty alcohols ranges from about 2% to about 8% by weight, based on the total weight of the composition, and
the weight ratio of the total amount of starch polymers to the total amount of cationic polymers is greater than 1.

22. The stable composition according to claim 7, wherein:
the total amount of electrolytes ranges from about 0.5% to about 3% by weight, based on the total weight of the composition,
the total amount of starch polymers ranges from about 0.75% to about 4% by weight, based on the total weight of the composition,
the total amount of cationic polymers ranges from about 0.35% to about 2% by weight, based on the total weight of the composition,
the total amount of fatty alcohols ranges from about 2% to about 8% by weight, based on the total weight of the composition,
the weight ratio of the total amount of starch polymers to the total amount of cationic polymers is greater than 1, and
the composition demonstrates no visible phase separation for at least about 4 weeks.

23. The method according to claim 20, wherein:
the total amount of electrolytes ranges from about 0.5% to about 3% by weight, based on the total weight of the composition,
the total amount of starch polymers ranges from about 0.75% to about 4% by weight, based on the total weight of the composition,
the total amount of cationic polymers ranges from about 0.35% to about 2% by weight, based on the total weight of the composition,
the total amount of fatty alcohols ranges from about 2% to about 8% by weight, based on the total weight of the composition,
the weight ratio of the total amount of starch polymers to the total amount of cationic polymers is greater than 1, and
the composition demonstrates no visible phase separation for at least about 4 weeks.
